(19) United States
(12) Patent Application Publication
(10) Pub. No.: US 2026/0166284 A1
(43) Pub. Date: Jun. 18, 2026

(54) GUIDEWIRE CONTROL DEVICES

(71) Applicant: BOSTON SCIENTIFIC SCIMED, INC., Maple Grove, MN (US)

(72) Inventors: ALEC SUNSHINE, Boston, MA (US); HANNAH ANDERSEN, Brookline, MA (US); DANIEL JOSEPH FAULKNER, Framingham, MA (US); JESSICA RINCON NIETO, Boston, MA (US); LAURA EMILY SABBAN, Norton, MA (US); NATHAN M. TENNEY, Leominster, MA (US); HAROLD D. HOVAGIMIAN, Acton, MA (US); ANITEZ GAUTAM, Morgan Hill, CA (US); JOHN THOMAS FAVREAU, Charlton, MA (US)

(73) Assignee: BOSTON SCIENTIFIC SCIMED, INC., Maple Grove, MN (US)

(21) Appl. No.: 19/424,064

(22) Filed: Dec. 17, 2025

Related U.S. Application Data
(60) Provisional application No. 63/735,487, filed on Dec. 18, 2024.

(51) Int. Cl. A61M 25/09; A61B 1/00

(52) U.S. Cl. CPC A61M 25/09; A61B 1/00131; A61M 2025/09083; A61M 2025/09116; A61M 2205/586

(57) ABSTRACT

Guidewire control devices and methods for making and using guidewire control devices are disclosed. An example guidewire control device may include a guidewire retention base having a channel formed therein. The channel may be configured to have a guidewire disposed therein during use. A guidewire securing member may be disposed along the channel. The guidewire securing member may be configured to releasably secure the guidewire to the guidewire retention base. A control region may be disposed along the guidewire retention base.

### CROSS REFERENCE TO RELATED APPLICATIONS

The present application claims the benefit of U.S. Provisional Patent Application Ser. No. 63/735,487, filed Dec. 18, 2024, the disclosure of which is incorporated herein by reference.

### TECHNICAL FIELD

The present disclosure pertains to medical devices, and methods for manufacturing medical devices. More particularly, the present disclosure pertains to guidewire control devices.

### BACKGROUND

A wide variety of medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. A guidewire control device is disclosed. The guidewire control device comprises: a guidewire retention base having a channel formed therein, the channel being configured to have a guidewire disposed therein during use; a guidewire securing member disposed along the channel, the guidewire securing member being configured to releasably secure the guidewire to the guidewire retention base; and a control region disposed along the guidewire retention base.

Alternatively or additionally to any of the embodiments above, the channel is arcuate in shape.

Alternatively or additionally to any of the embodiments above, the channel has an open side region.

Alternatively or additionally to any of the embodiments above, the guidewire securing member includes a guidewire securing magnet.

Alternatively or additionally to any of the embodiments above, the guidewire has a proximal magnet coupled thereto, the proximal magnet being configured to engage the guidewire securing magnet.

Alternatively or additionally to any of the embodiments above, the control region includes a finger ring.

Alternatively or additionally to any of the embodiments above, the guidewire retention base includes a biopsy cap.

Alternatively or additionally to any of the embodiments above, the control region is configured to secure the biopsy cap to a biopsy port of an endoscope.

Alternatively or additionally to any of the embodiments above, the biopsy cap includes a ratchet mechanism configured to allow at least a portion of the biopsy cap to rotate relative to the biopsy port.

Alternatively or additionally to any of the embodiments above, the guidewire securing member includes a surface texture.

A guidewire control system is disclosed. The guidewire control system comprises: a guidewire having a textured proximal end region; a guidewire control member configured to engage the guidewire, the guidewire control member including a guidewire gripping region; and wherein the guidewire gripping region includes a guidewire securing member disposed, the guidewire securing member being configured to releasably secure the guidewire to the guidewire control member.

Alternatively or additionally to any of the embodiments above, the textured proximal end region includes a coil and a braid disposed over the coil.

Alternatively or additionally to any of the embodiments above, the textured proximal end region includes a plurality of projections.

Alternatively or additionally to any of the embodiments above, the textured proximal end region includes an electro-spun coating.

Alternatively or additionally to any of the embodiments above, the textured proximal end region includes a guidewire magnet secured to the guidewire.

Alternatively or additionally to any of the embodiments above, the guidewire gripping region includes a gripping magnet configured to engage the guidewire magnet.

Alternatively or additionally to any of the embodiments above, the guidewire control member includes a biopsy cap configured to be releasably secured to a biopsy port of an endoscope.

Alternatively or additionally to any of the embodiments above, the biopsy cap includes a ratchet mechanism configured to allow at least a portion of the biopsy cap to rotate relative to the biopsy port.

A guidewire control system is disclosed. The guidewire control system comprises: a guidewire having a proximal end region; a guidewire control member configured to engage the guidewire, the guidewire control member including a base defining a guidewire gripping region; wherein the guidewire gripping region includes a guidewire securing member disposed adjacent to the base, the guidewire securing member being configured to releasably secure the proximal end region of the guidewire to the guidewire control member; and a finger ring disposed along the base.

Alternatively or additionally to any of the embodiments above, the proximal end region includes a guidewire magnet secured to the guidewire and wherein the guidewire gripping region includes a gripping magnet configured to engage the guidewire magnet.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a perspective view of an example guidewire control device.

FIG. 2 is a partial cross-sectional view of an example guidewire control device.

FIG. 3 is a perspective view of an example guidewire control device.

FIG. 4 depicts a portion of an example guidewire control device.

FIG. 5 depicts a portion of an example guidewire control device.

FIG. 6 is a perspective view of an example guidewire control device coupled to an endoscope.

FIG. 7 is a perspective view of an example guidewire control device.

FIG. 8 is a partial cross-sectional view of an example guidewire control device.

FIG. 9 is a side view of an example guidewire control device.

FIG. 10 illustrates a portion of an example guidewire.

FIG. 11 illustrates a portion of an example guidewire.

FIG. 12 illustrates a portion of an example guidewire.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

A variety of clinical interventions exist that use medical devices such as guidewires. Guidewire, in general, are elongated wires with a relatively small width or thickness. The size and dimensions of such guidewire may pose challenges for gripping and controlling the wire. Disclosed herein are guidewire control devices that may help a clinician manage guidewires, for example during clinical interventions.

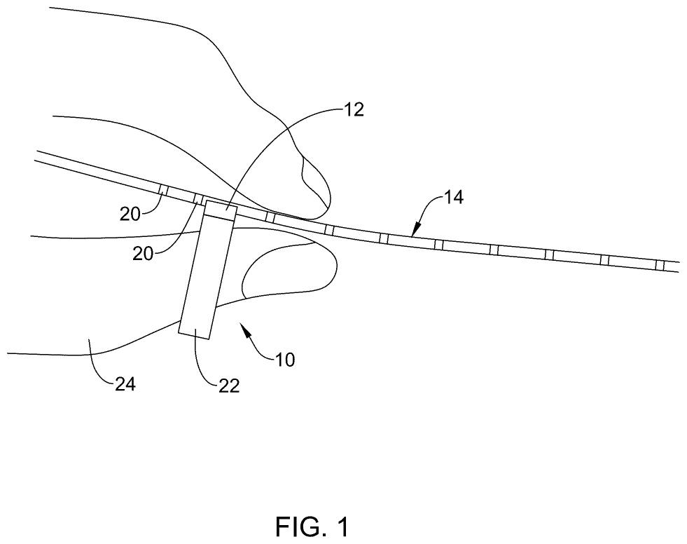

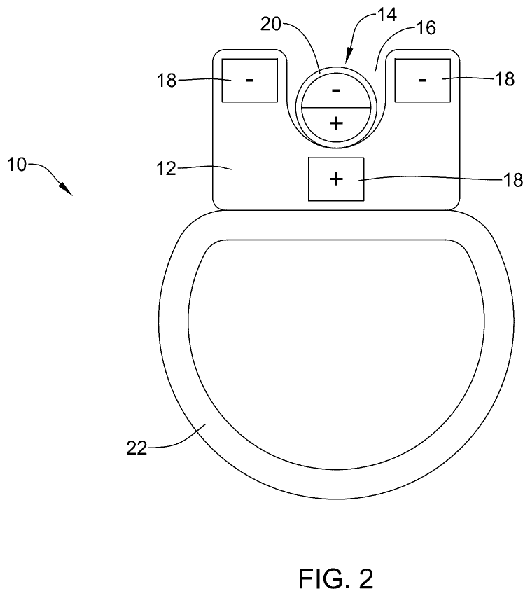

FIGS. 1-2 illustrate an example guidewire control device 10 generally configured to help a clinician manage a guidewire. In this example, the guidewire control device 10 may include a guidewire retention base 12. A guidewire 14 may be configured to engage or otherwise be coupled to the guidewire retention base 12. As shown in FIG. 2, the guidewire retention base 12 may include a channel or groove 16 formed therein. In at least some instances, channel 16 may be configured so that the guidewire 14 may be disposed therein. The shape of the channel 16 vary. In at least some instances, the channel 16 is generally arcuate in shape and may be open along a side region thereof (e.g., the channel 16 may have an open side region).

One or more guidewire securing members 18 may be disposed along the guidewire retention base 12 and/or along the channel 16. In this example, the guidewire securing members 18 may include one or more magnets (e.g., guidewire securing magnets). Other securing members are contemplated including textured surfaces, tacky coatings, and/or the like. In at least some instances, the guidewire 14 may include a guidewire magnet 20 (e.g., along a proximal end region thereof—e.g., a proximal magnet). The guidewire magnet 20 may take the form of an arcuate magnetic ring secured to the guidewire 14. Other forms are contemplated. It can be appreciated that the polarity of the guidewire securing members/magnets 18 and the guidewire magnet 20 may be arranged so that the guidewire 14 can be held in a desired manner. For example, the interaction of the guidewire securing members/magnets 18 and the guidewire magnet 20 may help to secured the rotational arrangement of the guidewire 14 and/or resist unintended rotation of the guidewire 14. In at least some instances, the guidewire securing members/magnets 18 and the guidewire magnet 20 may be arranged to allow for translation of the guidewire 14 (e.g., which resisting rotation). In FIG. 2, magnetic poles are indicated with either a plus (+) or negative (−) sign. The arrangement is not intended to be limiting as various arrangements are contemplated.

A control member or region 22 may be disposed along or otherwise be coupled to the guidewire retention base 12. For the purposes of this disclosure, the control region 22 may be understood as being a structural component of the guidewire control device 10 where a user may interface with the device in order to gain control of the guidewire 14. In this example, the control region 22 may include a finger ring that allows a finger or thumb 24 of a clinician to extend therethrough (see FIG. 1). The control region 22 may include a singular finger/thumb ring 22 or multiple rings configured to engage different fingers of a clinician.

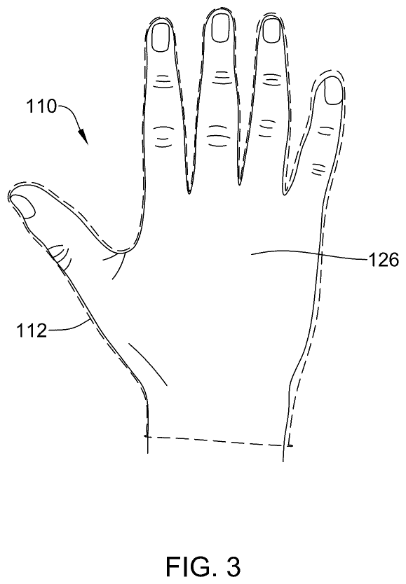

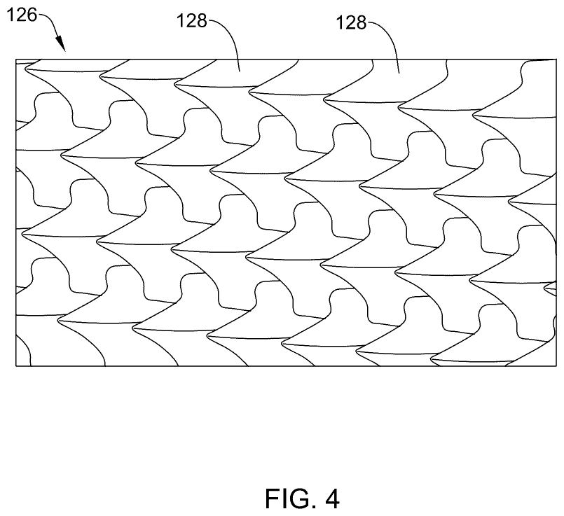

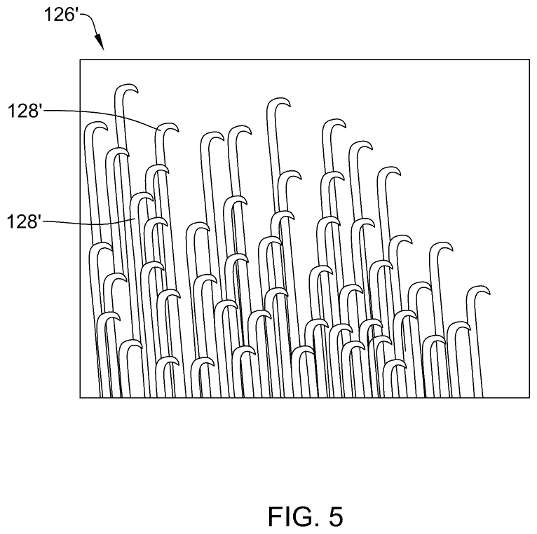

FIG. 3 schematically illustrates another example guidewire control device 110 that may be similar in form and function to other guidewire securing devices disclosed herein. In this example, the guidewire control device 110 may take the form of a glove 112. The glove 112 may be configured to fit over the hand (e.g. the entire hand) of a clinician. Alternatively, the glove 112 may be configured to be fitted over just one or more fingers (e.g., a finger glove). The glove 112 may have a surface texture 126 configured to enhance grip. The surface texture 126 may have a variety of configurations. For example, the surface texture 126 may have a form that resembles sandpaper, may resemble or include a low grad adhesive (e.g., with minimal residue), a high friction surface, etc. In one example, the surface FIG. 4 illustrates an example surface texture 126 embodied by a plurality of small projections 128. The projections 128 may form a complex array akin to a shark skin. In another example depicted in FIG. 5, an example surface texture 126′ is shown that may include a plurality of small projections 128′ that may be akin to a spider leg. These are just examples. Multiple surface textures are contemplated.

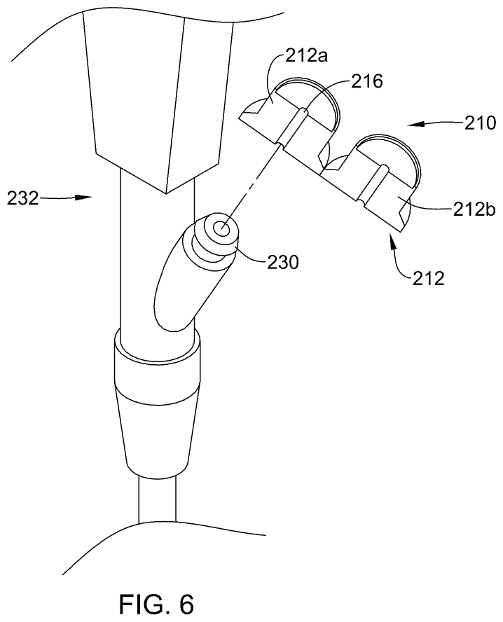

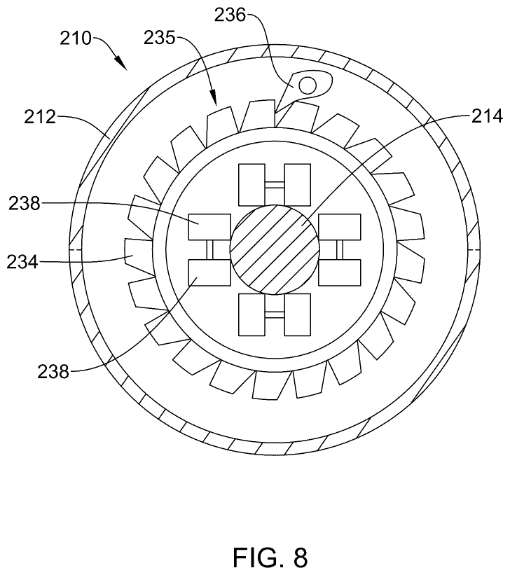

FIG. 6 illustrates another example guidewire control device 210 that may be similar in form and to other guidewire control devices disclosed herein. In this example, the guidewire control device 210 may include a guidewire retention base 212. The guidewire retention base 212 may include a channel or groove 216 formed therein. In at least some instances, channel 216 may be configured so that a guidewire (e.g., a guidewire 214 as shown in FIG. 8) may be disposed therein. The guidewire control device 210 may take the form of a biopsy cap configured to be secured to a biopsy port 230 of an endoscope 232.

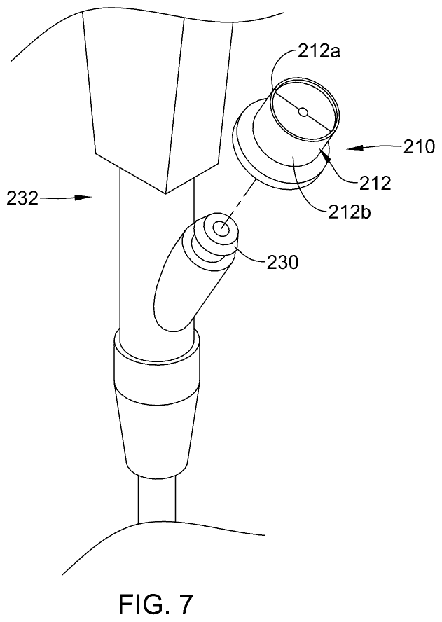

The guidewire retention base 212 may have a clamshell configuration where opposing halves 212a, 212b of the guidewire retention base 212 can be shifted between an open configuration (e.g., as shown in FIG. 6) and a closed configuration (e.g., as shown in FIG. 7). The open configuration may allow for a guidewire (e.g., a guidewire 214 as shown in FIG. 8) to be easily disposed therein. Once the guidewire is disposed in the channel 216, the clamshell can be closed onto the guidewire so that the guidewire can be suitable controlled.

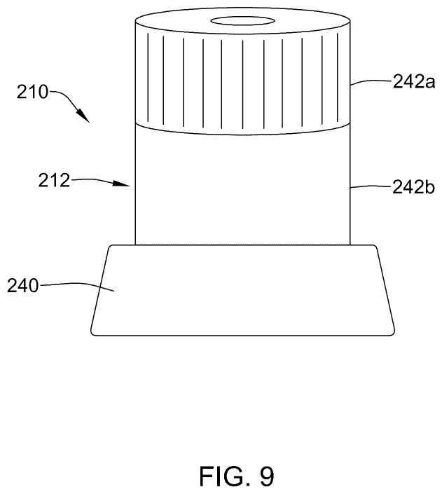

FIG. 8 is a partial cross-sectional view of the guidewire control device 210. Here it can be seen that the guidewire retention base 212 may include a ratchet mechanism 235 configured to allow at least a portion of the biopsy cap to rotate relative to the biopsy port 230. For example, the guidewire retention base 212 may be rotatable relative to a control region 240 of the guidewire control device (e.g., the control region 240 can be seen in FIG. 9). For example, the control region 240 may allow for the guidewire control device 210 to be secured to a biopsy port 230. The guidewire retention base 212, via the ratchet mechanism 235, may be free to rotate relative to the control region 240 and, thus, the biopsy port 230 when the guidewire control device 210 is secured to the biopsy port 230.

Referring back to FIG. 8, the ratchet mechanism 235 may include a gear 234 and a ratcheting pawl 236. As the guidewire retention base is rotated, the gear 234 may interact with the ratcheting pawl 236. This may provide tactile feedback to the clinician to indicate rotation has occurred. The ratchet mechanism 235 may also help to limit rotation to only a single direction and/or resist opposite rotation, which may further aid the clinician in maintaining control of the guidewire and/or keeping track of the rotational position of the guidewire 214. In other instances, the guidewire retention base 212 may be rotatable relative to the control region 240 in both directions. In other words, the guidewire retention base 212 may not be limited to rotation in only one direction and, instead, may be capable of rotation in either direction. In such instances, the guidewire retention base 212 may lack the ratchet mechanism 235 and/or one or more components thereof.

The guidewire retention base 212 may also include a plurality of rollers or wheels 238 (e.g., disposed along and/or helping to define the channel 216). The wheels 238 may be configured to engage a guidewire 214 extending through the channel 216. In some instances, the wheels 238 may include a rubberized, silicone, or high friction coating that helps to grip the guidewire 214 as the guidewire 214 passes through the channel 216. When the wheels 238 engage the guidewire 214, the guidewire 214 may spin with the guidewire retention base 212 (e.g., relative to the biopsy port 230). In other words, the wheels 238 may help to engage the guidewire 214 in such a manner that allows for the guidewire 214 to be held and rotated along with the guidewire retention base 212.

Referring back to FIG. 9, the guidewire retention base 212 may include a textured region 242a with a plurality of axially-extending ribs 245. The ribs 245 may help to enhance grip between a clinician and the guidewire control device 210. A number of different patterns of ribs 245 are contemplated including ribs that form a grid or mesh-like arrangement. In some instances, a portion 242b of the guidewire retention base 212 may be free of ribs. However, this need not be the case as the ribs 245 may extend the full length of the guidewire retention base 212.

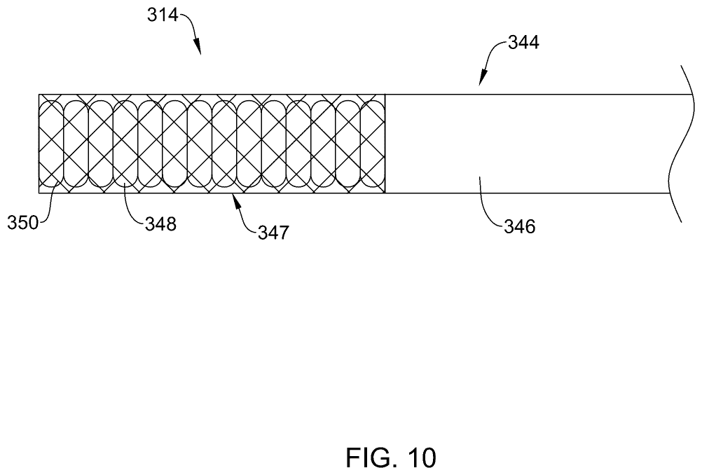

FIG. 10 illustrates an example guidewire 314 that may be similar in form and function to other guidewires disclosed herein. The guidewire 314 may include an elongate shaft or core 344 having a proximal end region 346. A textured region 347 may be defined along or adjacent to the proximal end region 346. The textured region 347 may include a coil 348 disposed along the proximal end region 346. In some instances, a braid 350 may be disposed over the coil 348. Alternatively, the braid 350 may be disposed along the proximal end region 346 in the absence of the coil 348. The coil 348 and/or the braid 350 may form a textured that can be felt by a clinician and allow for greater control of the guidewire 314 by the clinician.

The form of the coil 348 and braid 350 may vary. In some instances, the coil 348, the braid 350, or both may be formed from a polymer material. Alternately, one or both of the coil 348 and braid 350 may include a metal. A coating (not shown) may be disposed over the coil 348 and/or the braid 350. In some instances, the coating may include an electro-spun coating.

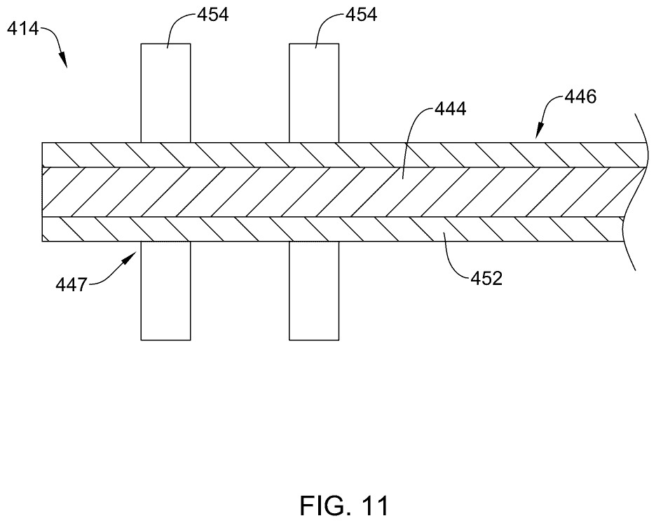

FIG. 11 illustrates an example guidewire 414 that may be similar in form and function to other guidewires disclosed herein. The guidewire 414 may include an elongate shaft or core 444. A textured region 447 may be defined along or adjacent to a proximal end region 446 of the shaft 444. The textured region 447 may include a coating 452 and a plurality of projections 454. The projections 454 may be integral with the coating 452 or may be separate from (and attached to) the coating 452. The projections 454 may take the form of bumps, dimples, rod-like structures, flaps, and/or the like.

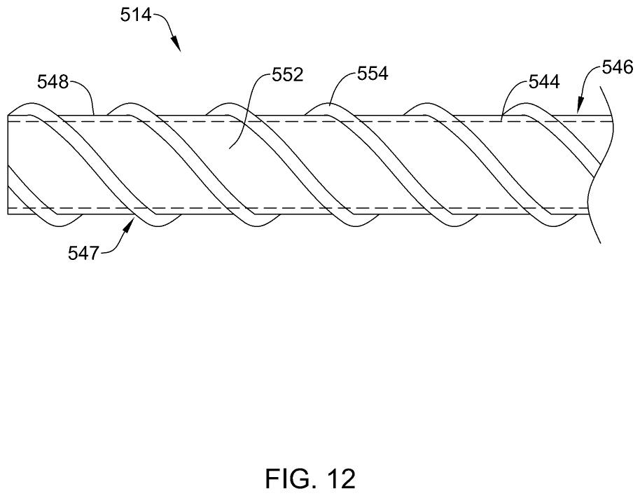

FIG. 12 illustrates an example guidewire 514 that may be similar in form and function to other guidewires disclosed herein. The guidewire 514 may include an elongate shaft or core 544. A textured region 547 may be defined along or adjacent to a proximal end region 546 of the shaft 544. The textured region 547 may include a coating 552 and a thread or coil 554 formed in the coating 552. The thread or coil 554 may be formed via an electro-spinning process. For example, the coating 552 may be applied to the shaft 544 via electro-spinning, and the electro-spinning process can be tailored such that coating process forms or defines the thread or coil 554 along the shaft 544.

The materials that can be used for the various components of the guidewire control device 10 (and/or other guidewire control devices disclosed herein) may include those commonly associated with medical devices. For example, the various components of the guidewire control device 10 (and/or other guidewire control devices disclosed herein) may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as VESTAMID®, GRILAMID® available from EMS American Grilon, and/or the like), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A guidewire control device, comprising:
a guidewire retention base having a channel formed therein, the channel being configured to have a guidewire disposed therein during use;
a guidewire securing member disposed along the channel, the guidewire securing member being configured to releasably secure the guidewire to the guidewire retention base; and
a control region disposed along the guidewire retention base.

2. The guidewire control device of claim 1, wherein the channel is arcuate in shape.

3. The guidewire control device of claim 1, wherein the channel has an open side region.

4. The guidewire control device of claim 1, wherein the guidewire securing member includes a guidewire securing magnet.

5. The guidewire control device of claim 4, wherein the guidewire has a proximal magnet coupled thereto, the proximal magnet being configured to engage the guidewire securing magnet.

6. The guidewire control device of claim 1, wherein the control region includes a finger ring.

7. The guidewire control device of claim 1, wherein the guidewire retention base includes a biopsy cap.

8. The guidewire control device of claim 7, wherein the control region is configured to secure the biopsy cap to a biopsy port of an endoscope.

9. The guidewire control device of claim 8, wherein the biopsy cap includes a ratchet mechanism configured to allow at least a portion of the biopsy cap to rotate relative to the biopsy port.

10. The guidewire control device of claim 1, wherein the guidewire securing member includes a surface texture.

11. A guidewire control system, comprising:
a guidewire having a textured proximal end region;
a guidewire control member configured to engage the guidewire, the guidewire control member including a guidewire gripping region; and
wherein the guidewire gripping region includes a guidewire securing member disposed, the guidewire securing member being configured to releasably secure the guidewire to the guidewire control member.

12. The guidewire control system of claim 11, wherein the textured proximal end region includes a coil and a braid disposed over the coil.

13. The guidewire control system of claim 11, wherein the textured proximal end region includes a plurality of projections.

14. The guidewire control system of claim 11, wherein the textured proximal end region includes an electro-spun coating.

15. The guidewire control system of claim 11, wherein the textured proximal end region includes a guidewire magnet secured to the guidewire.

16. The guidewire control system of claim 15, wherein the guidewire gripping region includes a gripping magnet configured to engage the guidewire magnet.

17. The guidewire control system of claim 11, wherein the guidewire control member includes a biopsy cap configured to be releasably secured to a biopsy port of an endoscope.

18. The guidewire control system of claim 17, wherein the biopsy cap includes a ratchet mechanism configured to allow at least a portion of the biopsy cap to rotate relative to the biopsy port.

19. A guidewire control system, comprising:
a guidewire having a proximal end region;
a guidewire control member configured to engage the guidewire, the guidewire control member including a base defining a guidewire gripping region;
wherein the guidewire gripping region includes a guidewire securing member disposed adjacent to the base, the guidewire securing member being configured to releasably secure the proximal end region of the guidewire to the guidewire control member; and
a finger ring disposed along the base.

20. The guidewire control system of claim 19, wherein the proximal end region includes a guidewire magnet secured to the guidewire and wherein the guidewire gripping region includes a gripping magnet configured to engage the guidewire magnet.
